(19) United States
(12) Patent Application Publication
(10) Pub. No.: US 2007/0269814 A1
(43) Pub. Date: Nov. 22, 2007

(54) METHOD OF PATHOGEN OR CHEMICAL DETECTION

(75) Inventors: JON G. WILKES, LITTLE ROCK, AR (US); DAN A. BUZATU, BENTON, AR (US); DWIGHT W. MILLER, WHITE HALL, AR (US); DANIEL L. CURTIS, NORTH LITTLE ROCK, AR (US); MARK DIGGS, LITTLE ROCK, AR (US); RAJESH NAYAK, LITTLE ROCK, AR (US); FATEMEH RAFII, WHITE HALL, AR (US); JOHN B. SUTHERLAND, WHITE HALL, AR (US); RANDAL TUCKER, HENSLEY, AR (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(73) Assignee: LITMUS, L.L.C., LITTLE ROCK, AR (US)

(21) Appl. No.: 11/558,586

(22) Filed: Nov. 10, 2006

Related U.S. Application Data
(60) Provisional application No. 60/736,116, filed on Nov. 10, 2005.

(51) Int. Cl. C12Q 1/06; C12Q 1/68; G01N 24/00; G01N 33/554

(52) U.S. Cl. 435/6; 435/39; 435/7.32; 436/173

(57) ABSTRACT

A method of determining the presence and level of microorganisms and/or chemicals in samples taken from generally any non-laboratory substance or environment. The method preferably comprises one or a combination of the steps of (a) prescreening for threshold levels of targeted microorganisms and/or (b) confirming the presence of targeted microorganisms or chemicals by mass spectrometry fingerprint analysis.

### FIELD OF THE INVENTION

The present invention relates to methods of pathogen and chemical detection, particularly for, but not limited to, samples taken from non-laboratory sources or environments which can contain particulates, multiple pathogens, and/or other contaminants.

### BACKGROUND OF THE INVENTION

A need presently exists for a method which will provide rapid pathogen detection wherein the presence and viability of bacterial cells can be determined in a matter of hours. The procedure will preferably be effective for detecting harmful levels of pathogens and/or chemicals in samples taken from non-laboratory sources or environments (e.g., food products, food processing facilities, medical patients, medical treatment facilities, sources of military or homeland security concern, etc.) which may contain particulates, multiple pathogens, and/or other hazardous agents or contaminants. A need particularly exists for a rapid procedure of this type which is accurate, selective, cost effective, and amenable to automation and is simple and rugged enough to be performed by lab technicians.

When conducting pathogen research and analysis in R&D laboratories, skilled researchers generally have the benefit of working with pure cultures and isolates in clean laboratory environments. They typically are also able to focus on a single target without having to contend with extraneous background particulate matter or the possible presence of multiple unknown pathogens or other agents or contaminants. Examples of products and procedures currently available in the art for use by skilled researchers for analyzing some types of laboratory cultures include: live/dead cell assay kits; antibody selective target tags; mass spectrometry fingerprint analysis and pattern recognition; ImmunoMagnetic Separation kits; mass spectrometry drift compensation; and sorting options using flow cytometry to characterize samples.

Unfortunately, when attempting to determine the presence and concentration of pathogens in samples taken from real world samples and environments, significant complications and barriers exist which typically prevent the use of straightforward laboratory procedures, techniques and suites. Examples of typical complications and barriers include: the presence of extraneous and/or unidentified background particulate matter; the possible presence of multiple unknown pathogens; the presence of other natural or added background substances (e.g., marinade compositions used in food products); and potential cross-reactivity issues between pathogens and reagents.

### SUMMARY OF THE INVENTION

The present invention provides a method for detecting pathogens and/or other hazardous agents which satisfies the needs and alleviates the problems discussed above. The inventive method is effective for detecting microorganisms and for detecting pathogenic levels of microorganisms in samples taken from any number of non-laboratory sources or environments. Examples of applications of the inventive method include, but are not limited to, food safety applications, medical diagnostic applications, and defense related applications. The inventive method is also effective for detecting and monitoring the presence of hazardous chemicals in the air, in water, or in other substances and environments.

In one aspect, there is provide a method of testing for microorganisms in a sample taken from a non-laboratory source or environment wherein the method comprises the steps of: (a) removing particulates from the sample; (b) determining whether at least a threshold level of viable cells, nonviable cells, or a combination thereof is present in the sample; and (c) determining, when at least the threshold level of viable cells, nonviable cells, or a combination thereof is determined to be present in the sample in step (b), whether at least one targeted microorganism is present in the sample.

In another aspect, there is provided a method of testing for microorganisms in a sample taken from a non-laboratory source or environment wherein the method comprises comprising the steps of: (a) removing particulates from the sample; (b) adding to at least a portion of the sample a first DNA-attaching dye of a type effective for attaching to DNA in viable cells and nonviable cells; (c) adding to at least a portion of the sample a second DNA-attaching dye of a type effective for attaching to DNA in the nonviable cells but which will not substantially penetrate into the viable cells; (d) determining a level of the viable cells and a level of the nonviable cells in the sample by flow cytometry based upon signal emissions of said first and said second DNA-attaching dyes; (e) adding to at least a portion of the sample a tag material effective for antibody selective attachment to a targeted microorganism; and (f) determining, at least preliminarily, whether at least a threshold level of the targeted microorganism is present in the sample by flow cytometry based upon a signal emission of the tag material.

In another aspect, there is provided a method of testing for microorganisms in a sample taken from a non-laboratory source or environment wherein the method comprises the steps of: (a) removing particulates from the sample; (b) adding to at least a portion of the sample a DNA-attaching dye of a type effective for attaching to DNA in nonviable cells but which will not substantially penetrate into viable cells; (c) adding to the portion of the sample a tag material effective for antibody selective attachment to a targeted microorganism; and (d) determining, at least preliminarily, whether at least a threshold level of viable cells of the targeted microorganism is present in the sample by flow cytometry based upon signal emissions of the DNA-attaching dye and the tag material.

In another aspect, there is provided a method of testing for microorganisms in a sample taken from a non-laboratory source or environment wherein the method comprises the steps of: (a) removing particulates from the sample; (b) recovering one or more cells from at least a portion of the sample by flow cytometry sorting; and (c) determining whether one or more cells recovered in step (b) is/are a targeted microorganism.

In another aspect, there is provided a method of monitoring air comprising the steps of: (a) concentrating particles of selected dimensions from the air; (b) placing at least a portion of the particles concentrated in step (a) into a liquid suspension; (c) analyzing the liquid suspension by mass spectrometry to obtain a spectral fingerprint for the particles; and (d) identifying the particles based upon the spectral fingerprint.

In another aspect, there is provided a method of monitoring air comprising the steps of: (a) capturing a chemical vapor in the air by filtration; (b) desorbing the chemical vapor captured in step (a) to produce a solution, vapor, or pyrolysate for analysis; (c) analyzing the solution, vapor, or pyrolysate by mass spectrometry to obtain a spectral fingerprint for the chemical vapor; and (d) identifying the chemical vapor based upon the spectral fingerprint for the chemical vapor.

In another aspect, there is provided a method of pathogen detection which uses the following instruments and methods, preferably in substantially the following sequence: (1) automated sample labeling and tracking methods (bar codes, etc.); (2) liquid handling robots; (3) batch sample cleanup by centrifugation and/or filtration; (4) cell viability assays by flow cytometry; (5) screening for targeted pathogens using fluorescence-tagged antibodies and flow cytometry; (6) immunomagnetic separation of target pathogens from non-pathogenic background bacteria preferably using an anchored antibody material selective for a targeted microorganism or for a genus, species, subspecies, serotype, or strain including the targeted microorganism; (7) small volume, batch culture of separated target bacteria to increase their number and standardize their growth conditions; (8) pyrolysis mass spectrometry of the grown target cells to provide a fingerprint for identification; (9) automated compensation of fingerprints for any distortions due to variations in cell culture conditions; (10) pattern recognition of the fingerprints (e.g., artificial neural pattern, multi-linear statistical pattern, expert system pattern, correlation analysis pattern, or other pattern recognition) for confirming target identification; and (11) automated reporting of results. In another preferred embodiment, steps (4) and (5) can be consolidated.

These steps provide rapid identification of pathogenic bacteria when present and allow even more rapid reassurance when they aren't. By using these methods commercial analyses can be completed rapidly and at very low costs. The prior art has not used flow cytometry techniques for applications outside a research environment nor does it facilitate correcting pyrolysis mass spectrometry (MS) spectral distortion for rapid commercial analysis. It doesn't include integration of these techniques into one system.

The inventive method for analysis of bacteria preferably comprises a suite of instrumental and computational techniques involving liquid handling robotics, flow cell cytometry, pyrolysis mass spectrometry, and computerized pattern generation, pattern drift compensation, and pattern recognition. Protocols are also preferably developed for each type of non-laboratory source or environment to be tested (e.g., food products, food processing facilities, medical patients, medical treatment facilities, water supplies, atmospheric air, etc.) which (a) account for the pathogens and other hazardous agents which could potentially be present in the particular source or environment and which (b) ensure that compatible fluorescent markers, antibody tags, and other agents and materials will be selected and used which prevent cross-reactions and other problems from occurring.

Provided below are several embodiments of the inventive method having in common the use of similar instrumental and computational sub-systems. For purposes of illustration, and not by way of limitation, the examples provided below are each optimized for a particular task, but they all preferably meet the following performance criteria:
Identification is rapid (compared to other competitive technologies; here this means reporting results in one to six hours compared to typical reporting in 24 to 48 hours);
Identification is as accurate and selective as required for the application intended for the particular embodiment (described below specifically for each).
The process is cost effective (with respect both to consumables and to time directly used operating expensive analytical instrumentation).
The process is readily automated for handling of batches comprising approximately 24 or more analytical samples each.
The process and sub-systems upon which analysis depends are rugged and simple enough for effective operation by a technician rather than a research scientist.

Further aspects, features and advantages of the inventive method will be apparent to those of ordinary skill in the art upon examining the accompanying drawings and upon reading the following Detailed Description of the Preferred Embodiments.

### BRIEF DESCRIPTION OF THE DRAWINGS

FIGS. 1-4 are flow charts illustrating the first embodiment of the inventive as described below.

### DETAILED DESCRIPTION OF THE PREFERRED EMBODIMENTS

Below are found several embodiments, each adapted for a particular application. It will be apparent to those in the art that the inventive method also includes any and all variations in specific elements such that the result of the variation is consistent with or advances the performance criteria listed above. It will also be apparent that the inventive method includes all other potential applications for bacterial identification in which such performance criteria are preferred.

A LA CARTE ANALYSIS OF TARGET STRAINS IN A FOOD PROCESSING PLANT. In one embodiment, the inventive method screens food samples to determine whether any of several classes of pathogenic bacteria (e.g.—Salmonella spp, Listeria spp, strains of Campylobacter jejuni, or E. coli O157:H7) are present, to quantify the concentration of each cell type, and to determine what proportion of bacterial cells present in each sample are viable.

The screening assays take from 30 minutes for a single analysis to one and a half hours for a batch of 96 analyses. (In the first case, the primary limiting factor is the time required for a biochemical reaction, described below. In batch analysis, this and other reactions are processed simultaneously.)

Sample Labeling: For handling large numbers of samples in a quality assurance/quality control (QA/QC) industrial production context, the inventive method preferably employs bar code or other sample identification labeling techniques, tracking of samples through the analytical process, and automated reporting of results for individual samples at intermediate stages of analysis followed by a summary report for each batch and archiving of results.

Initial Sample Preparation steps vary in sequence, number, and time depending on the particular food matrix or other sample: e.g.—a cell suspension rinsed from a lettuce leaf requires much less preparation than a cell and tissue suspension obtained from raw ground beef. Rapid sample preparation includes a sample-appropriate combination of selective centrifugation and coarse filtration steps designed to separate bacterial cells from food particles. This is accomplished using either individual centrifuge tubes and disposable, sterile syringe filters when there are a small number of samples, or four 24-well large volume filtering and non-filtering microtitre plates when preparing a batch of 96 or more samples. Consumable cost per analysis is reduced by using small liquid volumes and is further reduced drastically by batch operation. (24 analyses may use only two 24-well microtitre plates.)

Using a flow cytometer, rapid screening is possible because no cell culture or DNA amplification is involved. At the liquid flow rates typical in cytometry, single assays for viable or target cells can be completed in 15 to 60 seconds

Rapid cytometric screening for cell viability and total cell concentration is possible using two DNA-attaching fluorescent dyes. One of these is permeable to cell membranes and the other is not. If bacterial cells are present and viable, dyes that penetrate the cells concentrate in the cells' DNA, so the fluorescent color becomes much more intense in the cells than in the surrounding liquid. After adjustment to ignore the background fluorescent dye in the liquid, the cytometer will see and count cells based on the color emission of the first dye bound to their DNA. If a cell is not viable (membrane compromised) the second, originally impermeable dye now penetrates into its DNA and adds the second dye's signal to that of the first dye (binds to the DNA and emits a separate and distinct signal). The cytometer can count the number of viable and non-viable cells in an aliquot and calculate the concentrations and proportions of each.

Screening sensitivity for bacterial targets is based on antibody-selective attachment of fluorescent tags to the outside of target cells (30 minutes required for attachment reaction and 15 minutes for subsequent washing off of unbound antibody tags) and is essentially accurate to detect a single cell. However, counting cells of each target class takes variable amounts of time (15 to 60 seconds each) depending on the pathogenicity-determined thresholds for each target class and on the concentrations actually present in each sample. (Concentrated numbers of cells quickly exceed the threshold and yield a nominal positive assay, a result which then requires confirmation by an orthogonal rapid method, in this case mass spectrometry-based “operational fingerprinting”.)

ImmunoMagnetic Separation (IMS), like the target screening with fluorescence tagged antibodies, takes advantage of the antibodies' selectivity in adhering to target cells. In the case of IMS used for sample cleanup, there are no fluorescent tags. Rather, the antibodies are anchored to magnetic metal beads. By passing sample suspensions over and through the beads, only the targeted cells stick on the beads. The beads are washed to remove non-target cells or food debris. Then the remaining cells still adsorbed on the antibodies are desorbed to produce a suspension free from interference even though they came from a complex sample matrix.

Culture of desorbed cells before MS confirmation: A good-quality pyrolysis MS spectral pattern of bacterial cells is possible with as few 10,000 cells. Some bacteria produce symptoms when ingested in as a few as 10 viable cells per mL. Therefore, even with cell concentration there may not be enough target cells for confirmation. Also, since the cells at this point have not been growing under controlled conditions, their spectral fingerprints may not be accurate even if their number is sufficient to produce a good quality spectrum. For both of these reasons, the cells retained on the beads are often grown out in standard media under standard culture conditions. It is possible to grow more cells while the beads are still present. However, it is better practice that does not require excessive time to desorb the cells, centrifuge them to the bottom of the well and aspirate the supernatant, then reconstitute the cells in a non-selective, enriched liquid culture broth.

Target cells captured by the antibody-beads can be grown out directly without desorption from the antibodies or can be desorbed before growth. If they are desorbed, this should preferably be done in such a way that they sustain minimal damage from the process and thus remain viable, so they will grow quickly to provide the necessary minimum number. The time required for culture depends on the initial cell concentration. Analysis of the cultured cells by MS methods then provides good quality fingerprints free from spectral artifacts.

The reason for using a liquid broth rather than agar plates is so the grown cells can again be concentrated for rapid MS analysis. MS analysis by this method will be for a mixture of similar strains, not isolates. The spectral strains so analyzed will give an average MS fingerprint located somewhere in the region of spectral space occupied by the spectra of the various targets obtained from isolates. This approach does not require that the samples be isolated and thus saves the greatest amount of time. Confirmation of sample identity is obtained at the level of specificity associated with the antibody. If the antibody is genus-specific, so is the confirmation. If it is serotype specific, so is the confirmation. (If isolate-level identification is required for an analysis, this can be done using the rapid isolation and growout techniques described in Example 2 followed by pyrolysis MS.)

Rapid Mass Spectrometric “Fingerprinting” for sample identification is expedited by atmospheric pressure sample introduction so that acquiring each fingerprint takes as few as 10 seconds. Rapid turnover sufficient to lower cost per analysis cannot be easily achieved if samples are introduced individually through a vacuum lock into the instrument (several minutes per sample), as in conventional pyrolysis mass spectrometry designs. The mass spectrometer control computer acquires, averages, and processes spectral fingerprints, then associates each with the correct sample identity and analytical task, then exports each labeled spectrum to another computer for spectral drift compensation and pattern analysis. The time taken from initiating the MS acquisition to spectral export does not exceed 10 seconds per sample.

Drift-Compensation of Fingerprint Spectra for minor variations in experimental parameters is accomplished by identifying a combination of bacterial cells grown under the same variant conditions that can be used to track the changes that would occur for another, unknown strain. See “Drift Compensation Method for Fingerprint Spectra.” J. Wilkes, F. Rafii, K. Clover, M Holcomb, X. Cao, and J. Sutherland. U.S. patent application Ser. No. 09/975,530, filed Oct. 10, 2001. NIH (DHHS) Ref. No. E-169-00/0.

The computational process is written into a software packa that requires no expert judgment and produces drift-corrected spectra suitable for evaluation by a spectral fingerprint library in less than one millisecond per spectrum.

Drift compensated spectra are then identified, virtually instantaneously, by consulting artificial neural networks (ANNs) developed for each of the a la carte bacterial targets. An ANN for Salmonella spp. confirmation is based on drift-compensated spectra in a sub-library of isolate colony (single strain) spectra. The sub-library contains spectra for as many different Salmonella strains as necessary, including isolates obtained from the customer's own plant. Other entries in the spectral sub-library include representatives of non-Salmonella isolates typical for contamination in the customer's environment: e.g.—for a chicken processing plant, the other spectra include various Listeria spp., Campylobacter spp., normal E. coli, etc. Each sample nominally positive for Salmonella by the antibody fluorescent tag cytometry assay and containing an above threshold concentration of viable cells (another rapid cytometry-based assay), is analyzed for rapid confirmation by the combined MS, drift compensation, and ANN pattern recognition systems.

Results of both screening assays and MS confirmation are collated into reports which are electronically transmitted to the customer. The length of time to report generation depends on whether MS confirmation is required. If the screening assays are negative, reports to that effect are generated in 30 minutes to three hours. If positive, MS confirmation takes from three to six hours, depending primarily on the time required for sufficient cell reproduction in liquid culture.

### EXAMPLE 1

Exemplary Recipe: Screening and Confirmation of E. coli O157:H7 in hamburger meat.

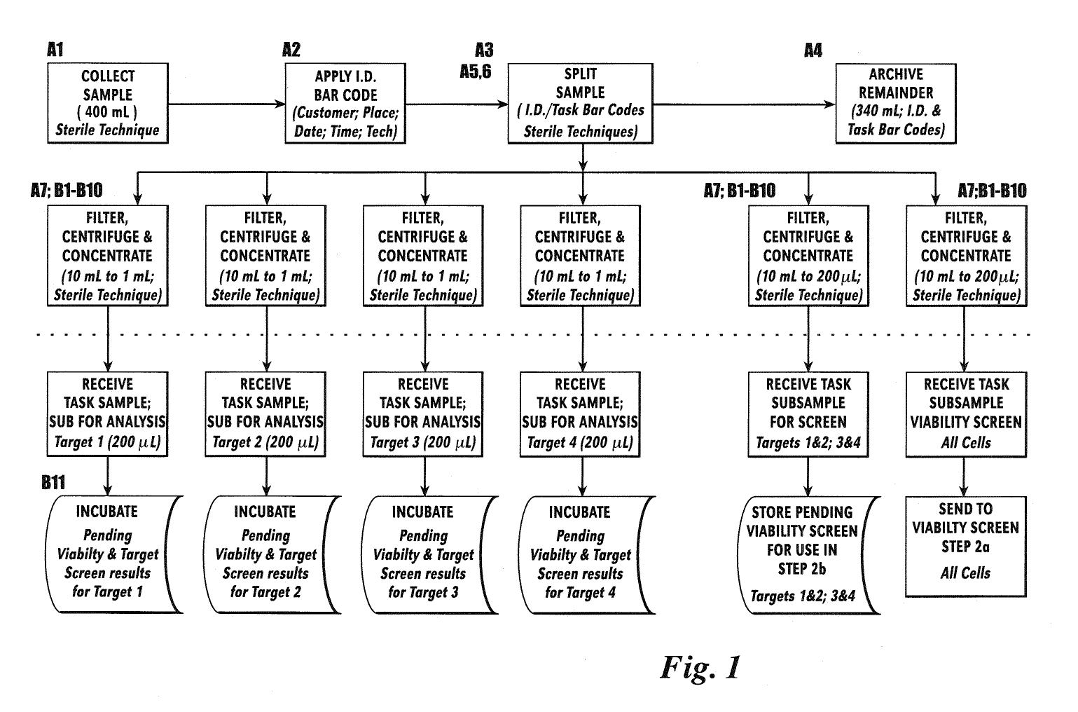

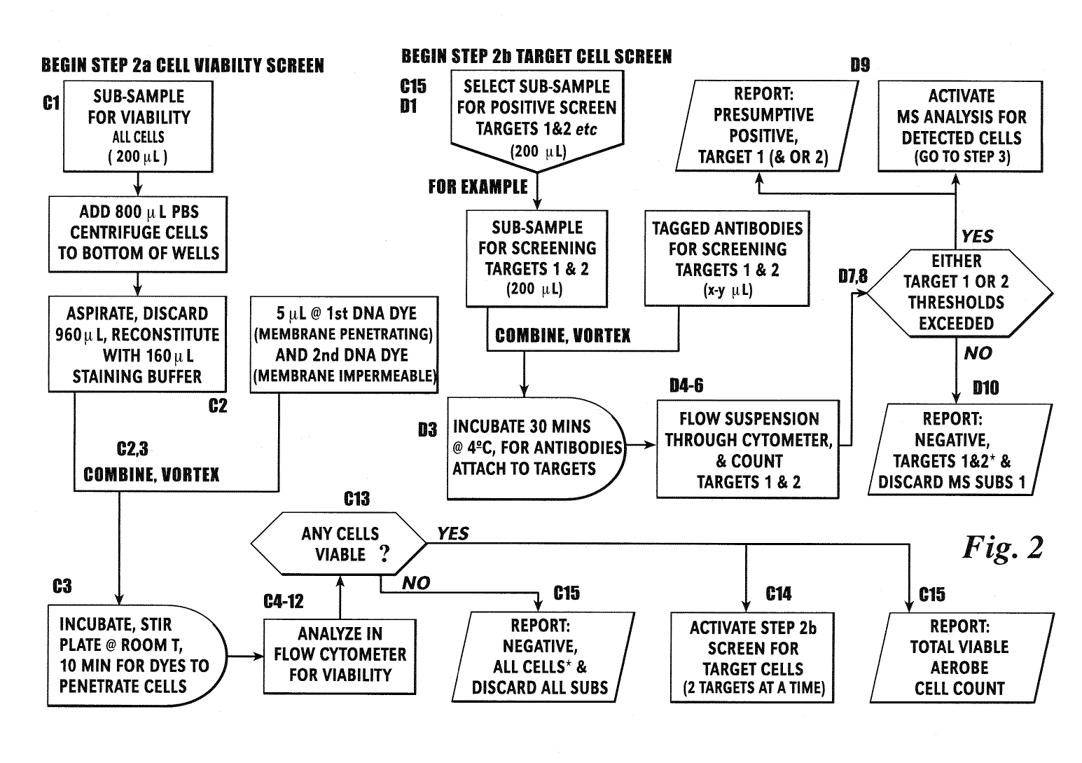

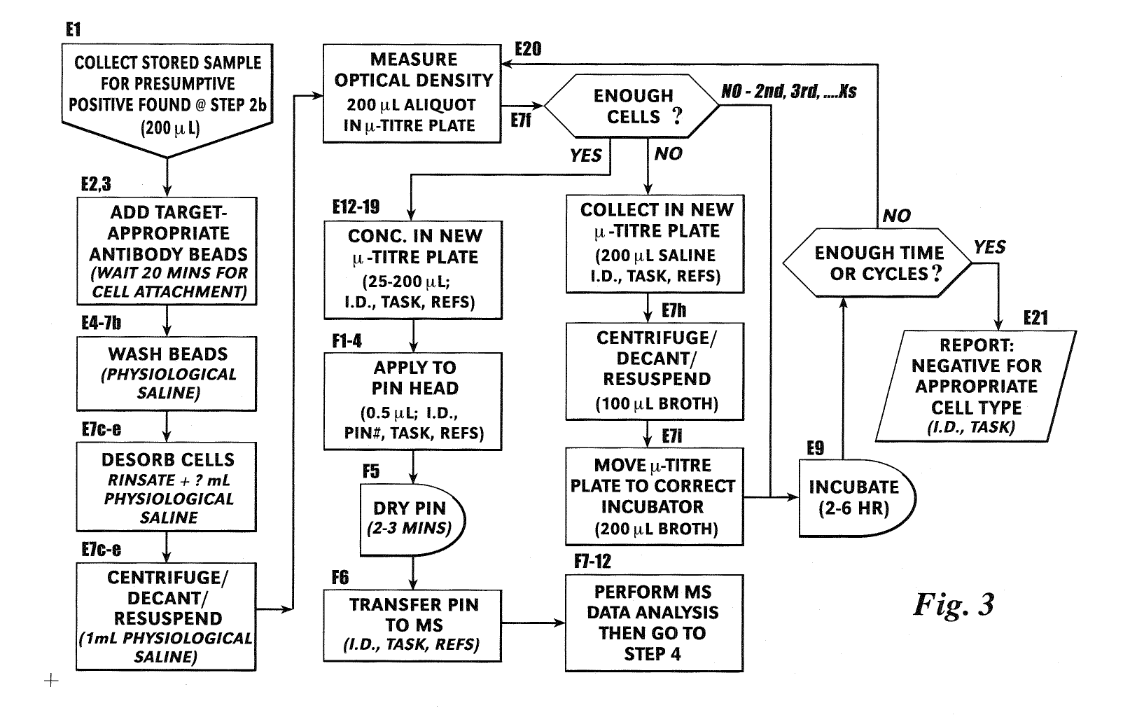

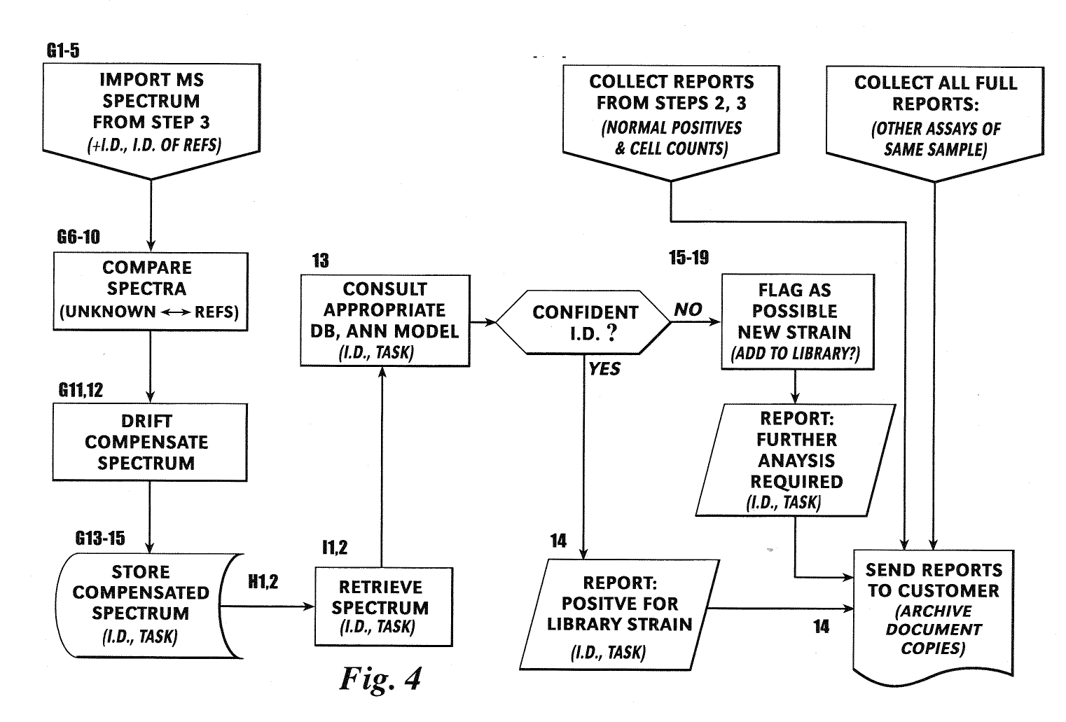

Alpha-numeric identifiers corresponding to the following steps are included on the flow charts provided in FIGS. 1-4.

A. Sample Labeling (Based on a 96 Analysis Batch)

1. (Customer or Testing Lab) Collect Rinsate Sample (typically 400 mL) using Sterile Technique and Store in a sealable, sterile plastic bag.
2. Apply to the bag an I.D. Bar Code containing this information: (Customer I.D #; Place,
   Date, and Time sample was taken; I.D. of the Technician who took the sample.)
3. Split the Sample into several 10 mL aliquots. (All sub-sampling uses sterile technique.)
4. Archive the Remainder in a freezer at the factory (for potential re-assays). Archived sample should also include a copy of each Task Bar Code (see below)
5. Affix to each sub-sample a copy of the I.D. Bar Code.
6. For each sub-sample, affix an appropriate Task Bar Code. In this example, the sub-sample task would specify “Analysis of E. coli O157:H7 for screening purposes” and another sub-sample would specify “Analysis of E. coli O157:H7 for confirmation purposes.” Similarly, other pairs of sub-samples would specify analysis for different bacterial targets potentially in the same sample.
7. Convey the two E. coli O157:H7 sub-samples (and 94 others) to a Sample Preparation Work Station: one or more Class II, Type A2 Biosafety Enclosure(s) housing two centrifuges and a liquid handling robot for microtitre plates; three small aerobic incubators set for 30°, 37°, or 42° C., respectively.
   B. Sample Preparation, common steps for both screens and confirmation (based on a 96 analysis batch) (Steps for a hamburger sample were adapted by combining a manual procedure published by Ochoa and Harrington, “Immunomagnetic Isolation of Enterohemorrhagic Esherechia coli O157:H7 from Ground Beef and Identification by Matrix-Assisted Laser Desorption Ionization Time-of-Flight Mass Spectrometry and Database Searches” Analytical Chemistry, 2005 with instructions for use of a filtering microlitre plate. Steps for rinsate from food samples are found in the accompanying FlowChart—Steps 1-4).ppt. In general, preparation of rinsate samples is less complex and quicker than for uncooked ground beef, which represents a food matrix of greater than average difficulty.)
1. 25 g of ground beef are weighed and placed in a filtered homogenizer bag (3M Microbiology, St. Paul, Minn.)
2. 225 mL of Phosphate Buffered Saline (PBS) is added and the contents stirred to homogeneity.
3. 1 mL aliquots of beef suspension are added to a tall 96 well microtitre plate and centrifuged at 2000×g for 10 seconds so that heavy debris settles to bottom and cells remain in suspension.
4. Using the robot, 200 μL of the supernatant in each well are transferred to a new, sterile 96-well filtering microtitre plate with an average 50 micron pore size.
5. Using the robot, 800 μL of sterile PBS is added to each well.
6. The filtering plate is stacked above a corresponding sterile non-filtering plate.
7. Bacterial cells and PBS can be pushed through the filtering plate by HEPA filtered air pressure or pulled through it using a vacuum collar situated below the filtering plate and above the plate receiving the filtered suspension. (The bacterial cells, but not larger particles of food debris, pass with the PBS through the coarse filter into the corresponding lower plate wells.)
8. Centrifuge the non-filtering plate at 5400×g for 5 mins.
9. Aspirate and discard 960 μL PBS supernatant (retaining the bacterial cells in the remaining 40 μL in each well)
10. Reconstitute the cell suspensions with 160 μL of either Staining Buffer (for Cell Viability Screen wells) or PBS (for Target Screen and MS Confirmation wells).
11. Place MS Confirmation microtitre plates into an incubator at the appropriate temperature for the various targets.
12. Mix suspensions in the incubating plate wells using a microtitre plate stirrer.
13. If cells are anaerobes, place the stirrer and plate inside specialty anaerobic atmosphere plastic bags inside the incubator.
   C. Sample Preparation and Conduct of the Cell Viability Screen
1. Prepare reagent solutions:
   8.1 μg/mL Thiazole Orange (TO) in DMSO;
   1.3 mg/mL propridium iodide (PI) in water;
   Staining Buffer: PBS, 1.3 mM ethylenediaminetetraacetic acid (EDTA), 0.0125% Tween-20 (Filtered through a 0.22 μm filter, use within two weeks).
2. Add 5.0 μL TO solution and 5.0 μL PI solution to 200 μL cell suspension in PBS:Staining Buffer (20:80) in each well of the 96 well microtitre plate.
3. Incubate with stirring using a microtitre plate mixer at room temperature for ten minutes.
4. Analyze on a flow cytometer (having 488 nm laser excitation, one forward scatter, one side scatter, and two fluorescence detectors set up for different wavelength emissions).
5. Set detection threshold or define intensity regions of interest (set a gate) to eliminate dilute fluorescence from unabsorbed TO and PI in solution.
6. Set PMT voltages in the Forward Scatter and Side Scatter detectors so that an entire population of unstained bacteria is entirely on scale in the forward scatter versus side scatter plot generated by commercial flow cytometers.
7. Emitted or scattered light is filtered so that only the frequency associated with TO is admitted to Fluorescence Detector 1 (FL1). Similarly, light admitted to FL2 is filtered so that frequencies in the range of PI emission pass to the detector.
8. A plot of FL1 versus FL 2 will show TO-stained bacteria in one region and PI and TO-stained bacteria in another. PMT values for these two detectors are set so that unstained bacteria appear in the lower left quadrant of an FL1 versus FL2 plot and a mixture of dead and lives cells stained with PI and TO, respectively, is completely on scale. Another way to describe this is that if the FL 1 versus FL 2 Plot is divided into quadrants, the dual-stained cells should appear in the upper right quadrant, the unstained should appear in the lower left and the TO-stained should appear in the lower right.
9. Define a gating strategy that counts separately cells appearing in the PI and TO associated regions of the FL1 versus FL2 plot. (A gating strategy is just a range of values for one or more parameters such that when an event occurs that falls within the rage (the gate), that event is counted as one of interest, whereas, events that fall outside the gate are not of interest and are ignored.) In this case, if three gates each comprise one of the aforementioned three quadrants in the plot, the instrument can be set to count the number of cells in each quadrant during acquisition.
10. Flow each of the 200 μL suspensions in the microtitre plate wells through the flow cytometer optical cell and count the number of TO-stained fluorescing (live) and PI-stained fluorescing (dead) cells appearing in their respective regions of the plot. (This will take a maximum of 2 minutes each at a 100 μL/min flow rate).
11. When the number of live cells exceeds the threshold for the most pathogenic target organism, flow can be terminated and the concentration of cells calculated from the proportion of the 2 minutes that was actually used.
12. If, unlike this example, the cells were concentrated during sample preparation by a factor of ten, for example), the true concentration of dead and live cells in the original extract or rinsate is 1/10^{th} of the number counted in this experiment. Rinsates being inherently cleaner than ground beef samples, more cell concentration is possible.
13. Compare counted or calculated concentrations to standard thresholds for target contamination (e.g. −10 viable cells/μL of extract or rinsate for E. coli O157:H7)
14. Mark for additional analysis the Target Screen Assays of samples in each well in which the viable cell count exceeds the relevant threshold(s).
15. Example: If a sample is being analyzing for E. coli O157:H7 with a threshold of 10 viable cells/μL and Salmonella with a threshold of 100 viable cells/μL and the preceding assay finds 50 viable cells/mL, then further screening for a Salmonella target is unnecessary. Report total viable cell count and a negative assay for Salmonella, mark to discard both the Salmonella target screen and the incubating MS confirmation subsamples, then proceed to Target Cell Screening for each possibly positive E. coli O157:H7 assay.)
   D. Sample Preparation and Conduct of Target Cell Screen for E. coli O157:H7
1. Reagents:
   anti-E. coli O157:H7 conjugated with a fluorochrome such R-Cy5 or fluorescein that absorbs at or near 488 nm and diluted in PBS/0.1% sodium azide/1% FBS by a factor of 1:100 to 1:10,000.
   PBS/0.1% sodium azide/1% FBS
2. Into the Target Assay microtitre plate wells, add 0.5 μL of diluted, fluorescence-tagged anti-E. coli O157:H7. (Antibody specific for other targets can be added for simultaneous analysis with E. coli O157:H7 if the fluorescence tags emit in a substantially different region, as was the case for TO and PI, above or as is true for R-Cy5 (680 nm) and fluorescein (530 nm). The number of different targets that can be screened simultaneously is limited by the lesser of the number of significantly different emission frequencies available from the fluorochromes and the flow cytometer's number of fluorescence detectors.)
3. Incubate antibody tag and cell suspensions in the dark at 4° C. with stirring using a microtitre plate mixer for 20-30 minutes.
4. After incubation, remove unbound antibody from the cells by washing with PBS/0.1% sodium azide/1% FBS. Add the wash, centrifuge at 350×g for 5-7 minutes at 2-8° C. Aspirate the supernatant leaving 5-10 μL in the bottom of each well. Repeat the wash two more times, if necessary.
5. Set detection thresholds, PMT voltages, and define regions of interest as in steps C4-C9 above.
6. Flow each of the potentially positive 200 μL suspensions in the microtitre plate wells through the flow cytometer optical cell and count the number of fluorescing cells appearing in the FL1 versus FL2 target defined regions of the plot. (This will take a maximum of 2 minutes each at 100 μL/min flow rate).
7. If necessary, use the inverse of any method concentration factor to correct the actual count for each target to its meaning in the original subsample suspensions.
8. Compare the corrected concentrations to threshold criteria for each target.
9. If the target cell number (whether alive or dead) exceeds that target's threshold concentration, report a presumptive positive and proceed to MS confirmation.
10. If the target cell number is less than the target's threshold, report a negative assay and mark all subsamples to be discarded.
   E. Sample Preparation (Using Immuno-Magnetic Separation, IMS) for MS-Based Confirmation
1. Reagents:
   anti-E. coli O157:H7-bead suspension (Dynal BioTech), Brown Deer, Wis.)
2. Into the MS microtitre plate wells, add 30 μL of anti-E. coli O157:H7-bead suspension to each 200 μL bacterial suspension corresponding to possibly positive results for E. coli O157:H7.
3. Incubate cell or bead/cell suspensions with stirring using a microtitre plate mixer at room temperature for 20 minutes.
4. Place microtitre plate into robot and set a permanent magnet plate along one side to pull bacteria-bead complexes that direction.
5. Use robot to aspirate all supernatant in each presumptively positive well.
6. Resuspend bead-antibody-bacteria complexes in 200 μL of PBS and proceed to step 7a or 7b.
7a. Repeat step 5 then proceed to step 8a or 8b.
   Alternative steps 7b-7g
7b. Use robot to transfer beads-antibody-bacteria complexes suspended in 200 μL PBS to a 24-well filtering microtitre plate with 50 μM pores placed above a non-filtering 24-well microtitre plate containing 100 μL NaOH buffer solution (pH 10) in each well.
7c. Place low positive pressure above the beads-antibody-bacteria complexes to force PBS through the filter but retain the complexes.
7d. Use the robot to transfer 130 μL aliquots of pH4 acetic acid or TFA buffer into each cell, to desorb cells from antibody-beads.
7e. Apply the pressure again to carry desorbed cells through the filter and into the neutralizing solution below.
7f. Measure optical density of desorbed cells to determine whether there are enough for immediate analysis.
7g. If there are, proceed to step E13.
7h. Centrifuge cell suspensions at 5400×g for 5 mins, aspirate supernatant, and reconstitute suspension with 100 μL TSB.
7i. Skip step 8, proceed to step 9.
8a. Resuspend bead-antibody-bacteria complexes in 200 μL of Tryptic Soy (or other target-optimal) broth (TSB).
   Alternative steps 8b-8e.
8b. Resuspend bead-antibody-bacteria complexes in 130 μL of pH4 acetic acid or TFA buffer to desorb cells from antibody-beads.
8c. Centrifuge antibody beads to the bottom of the wells at 2000×g for 10 seconds.
8d. Aspirate 100 μL cell suspensions, transfer into a new 96-well microtitre plate, and discharge the suspensions into 100 μL NaOH buffer solution (pH 10) in each well. (Note: steps 8b-8d preferably should be completed rapidly to minimize cell damage from the acid desorption wash)
8e. Centrifuge cell suspensions at 5400×g for 5 mins, aspirate supernatant, and reconstitute suspension with 100 μL TSB.
9. Place microtitre plate into incubator at 37° C. for E. coli [and all other aerobic targets except Listeria monocytogenes (30° C.) and Campylobacter jejuni (42° C.)].
10. Every hour remove the microtitre plate from the incubator, (set the permanent magnet alongside if following step 8a), and measure optical density in the relevant cells.
11. If optical density in wells doesn't meet threshold for MS analysis, return plate to incubator.
12. When optical density indicates sufficient growth for any of the wells, aspirate the suspension from those wells into another 96-well microtitre plate, and return previous 96-well plate to the incubator.
13. Centrifuge new wash/fixing plate at 5400×g for 5 mins, aspirate 70 μL of TSB, resuspend with 70 μL of PSB.
14. Repeat step 13 twice more.
15. Repeat step 13 but resuspend and fix cells with 50 μL of 70:30 ethanol:water or methanol:water.
16. Transfer contents from wells containing washed/fixed suspensions into corresponding wells in a 96-well MS storage sample plate.
17. As each well is filled with fixed cell suspension, the complete identity of the well's contents is transferred into the sample queue of the computer controlling MS acquisition.
18. To minimize evaporation, the MS storage sample plate is kept covered when not adding more samples to it or taking aliquots from it.
19. As more samples are grown, washed and fixed, cells are transferred into the corresponding positions in the MS storage sample plate and the cover is secured to minimize evaporation of fixing solvent.
20. Periodically the optical density is checked and steps 7f, 7h, 7i and 9 are repeated.
21. If in 6 hours, target cells do not grow enough to produce measurable optical density, report a negative confirmation for the target.
   F. Conduct of the MS-Based Confirmation Data (Using a JEOL AccuTofDART as MS Platform)
   Batch Setup: MS acquisition is set up for analysis of a batch of unknown samples thus:
      The Mass Spectrometer is changed from Standby to Setup Status.
      automated mass calibration and tuning of the MS is performed;
      continuous acquisition process is initiated by a QA/QC program. (This establishes Setup Status for the acquisition system.
      a batch initiation message with batch identification is exported to a Batch Processing PC (BPPC)
      a representative background spectrum is acquired, stored in the acquisition computer's (AcPC's) own memory registers;
      acquisition using the same process as described in steps 1-10 below, of a series of reference pyrolysates for known bacterial isolates (however, these spectra are designated by their bacterial strain as well as the batch data and series, they represent; they also have an <*.ref> extension that clearly marks them as reference rather than unknowns.
      export of these reference spectra from the AcPC to the BPPC, for later use by a spectral drift compensation algorithm.
1. When ready for MS analysis, the 96-well MS storage sample plate is removed from the receiving location of the liquid handling robot and placed in the loading position of the MS pin loading robot: e.g.—for loading suspensions onto MS pyrolysis pins.
2. Clean MS pyrolysis pins are stored vertically protruding from an aluminum block. Pin holes in the block are found in a 8×12, 96-hole array that mirrors in spacing and dimensions the overall shape of a typical 96-well microtitre plate.
3. The pin holder block is located in the receiving position of the pin loading robot.
4. The robot stirs the contents of a designated well in the storage plate, then samples a 0.5 μL aliquot and deposits it on the head of the corresponding pin.
5. Two to three minutes are allowed to ensure evaporation of the fixing solvent so that only cells and dissolved non-volatile extracts remain: the biochemicals that will define the MS “fingerprint” pattern.
6. When all loaded pins are dry, the pin holder block is removed from the pin loading robot stage and relocated on the MS autosampler stage and the MS is placed in Operate Status.
7. In the order that the sample identities were queued (step E17, above), loaded pins are robotically transferred from the autosampler stage to the MS sample introduction gear assembly.
8. On command either manually or automatically from the AcPC, the pins are rotated by the sample introduction gear, 90 degrees from the vertical “Load” position, to the horizontal “Pyrolysis” position.
9. Pyrolysis is initiated for 3-5 seconds achieving a maximum temperature of 500° C. on the pin head. (This rapid heating is achieved by passing 10 amps at 12 volts DC through a ⅛ inch length near the end of a 1/16^{th} inch diameter pin.)
10. In five to nine seconds, the AcPC operating the MS acquires signals from all pyrolysates, averages the spectra, subtracts background, identifies MS peaks at high resolution, automatically attaches the sample identification information from the queue, and exports all of this information to the BPPC as one relatively small (50 KByte) ASCII file for each analysis. The file format is “Standard Format Header” followed by a two column (High Resolution Mass, Intensity) spectrum.
11. After exporting the data, the AcPC automatically clears the sample spectrum region of the memory registers of the sample data (but not the average background spectrum) and resets, awaiting the next manual or automatic acquisition command for the next sample in the queue.
12. When the batch is complete, the AcPC transmits a batch termination code to the BPPC, clears that batch's background spectrum from its own registers and returns to Setup Status for the next batch (or Standby Status at the end of the day or Shutdown Status for MS maintenance/repair).
   G. Drift Compensation of Mass Spectral Patterns

As described above, high resolution, background-subtracted, peak-identified pyrolysis mass spectra are imported into the BPPC for all Reference and Unknown spectra in a batch. (The batch is defined in the BPPC by Initiating and Naming commands and by a Batch Termination code.)

The BPPC contains a Processing Folder, the drift compensation module (DCM, an executable program), batch specific folders for archiving uncompensated spectra, a folder containing a sub-library of customer-specific spectra and relevant entries imported from the Litmus Global Spectral Library), a Temporary Storage Folder for drift-compensated spectra produced during current operations on data in the Processing Folder, an archive of folders for each batch of drift-compensated spectra, a Temporary Folder for Reports of the Batch in Progress, and an archive containing customer folders for final reports of each batch (for billing and other business purposes).

The drift compensation process below is an automated realization of the concepts disclosed in “Drift Compensation Method for Fingerprint Spectra.” J. Wilkes, F. Rafii, K. Glover, M. Holcomb. X. Cao, and J. Sutherland. U.S. patent application Ser. No. 09/975,530, filed Oct. 10, 2001. NIH (DHHS) Ref. No. E-169-00/0.

Detailed Procedure:

The steps for spectral drift compensation follow:

1. Import batch identity from AcPC into the BPPC Processing Folder.
2. As they are acquired, import from the AcPC each Headed Spectrum (Batch Reference or Unknown Sample) into this same Batch Processing Folder.
3. Copy the Processing Folder contents to the corresponding Batch Archive Folder with auto-update after each new spectrum or other data packet is received.
4. DCM operation is initiated by AcPC command after batch identification.
5. Upon initiation, DCM queries the contents of the Processing Folder every five seconds and uploads any new items.
6. As each reference spectrum is received it is divided by all corresponding replicates (suppose NR=5) in the Spectral Library and the dividends are arrayed into an m/z×5 correction factor matrix particular for that reference in the batch.
7. The corresponding entries in each row of the correction factor matrix are averaged to generate an m/z×1 average correction factor matrix.
8. Steps 6 and 7 are repeated for each reference spectrum until all references have been analyzed to determine their average correction factor matrix.
9. As soon as an unknown sample spectrum arrives at DCM, the Euclidean distances between it and each of the previously acquired reference spectra in the batch are calculated and stored as a Distance Matrix.
10. The multiplicative inverse of the sample's Distance Matrix is normalized so that the sum of all entries is 100. This is that sample's Reference Weight Matrix. (This gives the greatest weight to reference samples at the shortest distance from the unknown.)
11. The unknown spectrum is multiplied by each Average Correction Factor Matrix to generate a Matrix of Provisional Drift-Compensated Spectra for that unknown, each column representing one particular type of reference.
12. The various provisional corrected spectra (columns in the Provisional Drift-Compensation Matrix) are weight-averaged, with weights designated by the corresponding Reference Weight Matrix, to generate a single drift-compensated spectrum, designated as such using a <*.dcs> terminal file name extension.
13. This drift-compensated spectrum is copied to the Temporary Storage Folder and also archived in the corresponding folder of <*.dcs> files reserved for all unknowns in that batch.
14. As each unknown spectrum is imported, steps 9 through 13 are repeated.
15. When the Batch Termination Code has been received in the BPPC Batch processing Folder, DCM terminates the batch by transferring all *.dsc files from the Temporary Storage Folder to that Batch's Drift-Compensated Archive.
   H. ANN Model Creation Based on Validated Entries for Isolates in a General Pyrolysis Mass Spectral Library.
1. Use the following process for conflating into <800 bins the high resolution PyMS spectra of a typical training set defined for identifying a particular target (e.g.—a set for identifying E. coli O157:H7, which would include many E. coli O157:H7 strains, several other E. coli of different serotypes as well as Shigella spp. and some other genera and species):
   a. Compile all training set spectra into a group.
   b. List by m/z all values that appear in at least one of the spectra in the training set group. (Define ion peaks from different spectra as belonging in the same bin if their m/z values are within 0.01 to 0.05 amu of each other [or for a standard that varies with ion size] 1 to 5 ppm of the m/z value.)
   c. If the resulting set includes more than 800 bins, automatically scan the set for the MS peak with lowest relative intensity, eliminate that peak and query the number of bins that would be generated by repeating step 1b.
   d. Continue serial elimination of low intensity peaks until only 800 bins remain.
2. Using the training set spectra, build and cross-validate an artificial neural network (ANN) model having <800 nodes in its input layer, the experimentally determined optimal number of nodes in its hidden layer, and the number of nodes in its output layer corresponding to the number of strains in the training set.
   I. Consultation of Pyrolysis Mass Spectral Libraries for Unknown Identification
1. Ignore any ions found in an unknown spectra that are not within 0.01 amu of the bins used to define the training set for the ANN appropriate for the particular type of target strains in the batch.
2. Take the <*.dcs> spectra for each unknown, with the extraneous ions removed, normalize it to the same total intensity standard as used for the spectra in the training set.
3. Interrogate its identity using the appropriate trained and validated ANN model.
4. If the strains with highest probability are of the E. coli O157:H7 type, report confirmation of sample identity to the customer and complete all other appropriate archiving and sample disposal steps.
5. If none of the strains has a high probability of belonging to any of the ANN categories, report an ambiguous or negative confirmation of identity and flag this data for expert QA/QC evaluation.
6. If upon expert examination of the spectra, there is no obvious reason to believe the analytical process was flawed (e.g.—pyrolysis was actually conducted and the spectrum looks like a typical bacterial pyrolysis spectrum), then resample from the MS storage sample plate and reanalyze or begin analysis again from archived samples.
7. If re-analysis confirms a similar result, use these drift compensated spectra with the larger spectral library and more conventional (but slower, non-automated) multilinear pattern recognition techniques to query whether the sample appears to belong to another strain in the library not used in the ANN training set.
8. If the sample can be identified by this process, confirm the identity and report it to the customer as the result corresponding to the presumptive positive in the screening tests. Also, add the newly found strain to the library and to those used to define the ANN training set for this type of target analysis. Then rebuild and revalidate the ANN model by the techniques listed in part H. above.
9. Whenever a sample is identified conclusively using steps 7-8, add the newly found strain to those used to define the ANN training set for this type of target analysis. Then rebuild and revalidate the ANN model by the techniques listed in part H. above.
10. Whenever a sample is not in either the ANN training set or the general spectral library, live cells from the sample should be saved and subject to isolation and testing by the usual panel of microbiological assays.
11. If after isolation, the sample appears to contain a single new strain or a group of new strains, each new isolate from the sample should be saved and spectra obtained with drift compensation for addition to the general library and to an improved ANN training set.

RAPID ISOLATION AND/OR RAPID CONCENTRATION. In another embodiment of the inventive method, the same kinds of instrumentation can be used for accelerated isolation and identification of target and non-target strains found in unknown samples. The process is similar to the descriptions in Example 1 above but it
does not necessarily require the use of any antibody fluorescent tags but
does require a more sophisticated flow cytometer, one equipped with
   a forward scatter detector sensitive for bacterial cells of 1-3 μM length and
   an optional attachment capable of sorting individual cells into small volumes of culture broth in individual wells of a sterile, 96-well microtitre plate.
      Using this instrumentation, rapid isolation of bacterial cells in an unknown cell suspension is not difficult. The major technical challenge is to calibrate the flow cytometer's cell sorting option so that:
one and only one cell in a stream is identified as such (e.g.—not a lump of tissue or even a cluster of bacterial cells, which might be of different strains), and
the path of the droplet containing that cell is controlled so that the selected droplet (and no other) is propelled into the selected well in the 96-well plate, and . . .
all non-selected droplets are sanitized and passed out of the system to waste.

### EXAMPLE 2

Detailed Recipe:

Major Steps A and B are the same as in Example 1.

If desired, Major Step C in Example 1 can also be followed to confirm the presence of cells and that some of them at least are viable, though this is not necessary for this application.

Major Step D steps 1-5 in Example 1 may also be followed if one desires to isolate only those cells associated with a particular target type. In this case, operation of the sorting option by the process disclosed in this Example would be indicated only for isolated cells exhibiting the specific color fluorescence associated with the fluorescence tag.

D. Flow Cytometry Sorting Option for Use with 96-Well Plates: General Capabilities.

The sorting option can be used to concentrate a counted number of untagged cells of a distinctive morphology in the presence of other untagged cells lacking the distinctive morphology. For example, to separate bacilli (rods) from coccuses (spheres) or to separate bacillus spores (dense rods) from bacillus vegetative cells (less granular rods but with the same shape and size as the corresponding spore). Another sorting action can allow separation of a single cell for purposes of rapid isolation. The physical operation is similar to concentration except that the allowed cell count per well is set from, say 20,000 that meet sort criteria, to one (1). Also, in cell isolation sorting, a criterion called pulse-pile-up (PPU) is activated so that a droplet is not chosen for sorting when it contains more than one bacterial cell of the proper size and shape. PPU and the sorting option together assure that the cell suspension resulting from subsequent culture within the well will be a pure isolate, because all cells in the suspension were grown from the sorted one.

E. Calibration of the Flow Cytometry Sorting Option for Use with 96-Well Plates

1. High voltage is deactivated and the sort option arm (which holds and moves the 96-well microtitre plate during sorting) is extended from its home position to accept the microtitre plate holder.
2. The multiwell plate holder is installed on the arm and perform autocalibration (which automatically determines the exact location of the microtitre plate holder in relation to the arm and other mechanical components of the system).
3. Install a slide adapter on the plate holder in order to set up for an operation called the sort matrix. Sort matrix will calibrate the exact delay time between detected microbial cells by their laser light scatter or fluorescence emission and initiation of a voltage to deflect the droplet that contains them into a selected well in the microtitre plate.
4. Turn on the sheath and setup sort streams and adjust the left sort stream so that it is vertical. Toggle the deflection high voltage on and off to assure that the left sort stream looks the same as the sheath stream when high voltage is off.
5a. Manually calculate the approximate time and distance between laser actuated observation of a bacterial cell and the top of the electrical deflection grid. Time and distance depend on the liquid flow rate, forward velocity, channel diameter and component spacing inside the cytometer flow cell. In practice the distance and time are measured as the number of droplets (or incipient droplets) that will pass through the flow cell before an observed bacterial cell arrives at the electrical deflection plate. This number is a function of the liquid velocity, distance, and vibration frequency (e.g. −˜32 kHz) that determine droplet volume. The number of droplets is also called the prop Delay and can vary from 5 to 65 drops. It will be assumed for purposes of this example that the number comes out as 48.
5b. Alternatively, use the flow cytometer's Sort Matrix to determine the approximate prop Delay. This is done by physically observing the stream as it emanates from the flow cell and begins to break into droplets as it approaches the charge plate region. One can observe this using a television camera because the droplets are lit using a stroboscope timed at the same frequency as the flow cell vibrations. This visually “stops” the droplets and the operator can see on a TV monitor exactly where the stream begins to form fully detached droplets. For accurate sorting it is critical that the last (barely) attached droplet be located at the entrance to the charging region. By adjusting liquid flow rates, vibration frequency, or other parameters it is possible to position the last attached droplet exactly and also determine the drop delay.
6. A sample of fluorescently labeled spheres of about the same size as bacterial cells is run through the flow cytometer and a sort region is defined to count these spheres as they pass through the system.
7. Sort Matrix operation is selected, the approximate prop Delay previously estimated is entered as well as a prop Step Delay value. This step value determines how many steps either side of estimated value the Sort Matrix will automatically check to see which value is actually correct. In this example, if the Step Delay is four and the estimated prop Delay is 48, the Sort Matrix will check prop Delays from 44 through 52.
8. A number of acceptable sort “events” (e.g.—with PPU turned on, single fluorescent beads observed in a droplet) is selected. This number could be 20, for example.
9. A clean microscope slide is placed into the slide adapter slot on the plate holder and the Sort Matrix is started.
10. The Sort Matrix places the slide where one spot the size of a 96-well plate well will catch the selected events from the 44^{th} drop.
11. The Sort Matrix counts 20 events, then moves the plate holder to the next “well” position. Again 20 events are counted but the sorting is performed on the 45^{th} drop.
12. This sequence is continued until all the Drop Delays from 44 to 52 have been checked and the operation is stopped.
13. The slide is removed and each “well” location is checked under a fluorescence microscope and the actual number of beads is counted in each.
14. One or two of the “well” locations will have the closest to 20 beads. Let us suppose here that the 47^{th} droplet gave us 19 beads and the 48^{th} gave use 18. Typically, if the basic operation of the cytometer has been properly adjusted, the other wells for longer or shorter drop delays will be almost or completely free of beads. From these observations the operator knows within one droplet what the optimal Drop Delay should be.
15. A similar set of experiments is then conducted using fractions of a drop over the range between 47 and 48. In these experiments one will count almost 20 beads in each, but perhaps drop delay 47.4 gives exactly 20 beads.
16. The cytometer is set for a Drop Delay of 47.4 and is now calibrated for cell sorting.
   F. Use of the Flow Cytometry Sorting Option for Culturing Bacterial Isolates within 96-Well Plates
1. The Drop Count is reduced from 20 to 1.
2. The Cytometer sort region is redefined from that for beads to one appropriate for the desired fluorescence region (tagged cells) or forward- and side-light scattering characteristics (untagged cells).
3. A sample from which isolates are to be obtained is run and a user-defined number of such isolated cells are deposited, one cell per well, into a series of microtitre plate wells containing TSB or some other culture medium appropriate for the cells of interest.
4. When the cells have been filled, another series of wells can be filled for the next sample by repeating steps 2 and 3.
5. When all wells are filled or all isolation operations have been completed, the microtitre plate is covered, removed from the cytometer, and transferred to the appropriate incubator for growth.
   G. Use of the Flow Cytometry Sorting Option for Concentrating Selected Cell Forms within 96-well Microtitre Plates
1. To perform cell concentration the Drop Count is changed from 20 to perhaps 20,000.
2. The Cytometer sort region is redefined from that for beads to one appropriate for the desired fluorescence region (tagged cells) or forward and side light scattering characteristics (untagged cells).
3. A sample for which selective concentration is desired is run and, in this example 20,000 droplets with 20,000 selected cells are added to each well containing TSA, PSB or ethanol fixing solution, depending, respectively, on whether the cells are to be immediately grown up, partitioned for multiple operations, or immediately analyzed in an MS.
4. When the cell has been filled, another well can be filled for the next sample by repeating steps 2 and 3.
5. When all wells are filled or all concentration operations have been completed, the microtitre plate is covered, removed from the cytometer, and (depending on the solution used in step 2)
   transferred to the appropriate incubator for growth, or . . .
   subject to further manipulations of the viable cells, or . . .
   transferred to the MS as dead cells for rapid identification.
      Comprehensive Detection and General Classification of Chemical or Biological Materials in a Generic Context, such as Environmental Air- or Water-Quality Monitoring, where there is not Necessarily a Basis for Anticipating Particular Analytical Targets (Other than the Air or Water).

A principal and significant advantage of mass spectrometers used as detectors is their potential for identifying most substances, biological or chemical. The following embodiment exemplifies this capability through an air-monitoring example in which there is no a priori assumption about the biological or chemical nature of substances of interest.

The environmental air monitoring system includes a battery of virtual impactors that concentrate aerosol particles of selected dimensions from the air onto small targets as well as, downstream of the impactors, activated carbon or other high efficiency filters that capture and concentrate airborne chemical vapors. The concentrated particles from the virtual impactor are sampled into a liquid suspension or solution for analysis by deposition and evaporation on the head of a pin as in the MS confirmation process of Example 1. The filters are chemically desorbed to produce a similar solution for subsequent analysis as in Example 1 via the same route. Alternatively, the thermally desorbed vapors are analyzed directly by the mass spectrometer. Volatiles in the ambient air can also be analyzed without concentration or filtering. In all four cases, detection and identification generally track the mass spectrometric and pattern recognition procedures already described in Example 1.

Samples of this sort are typically not chemically pure. However, they may be highly concentrated in certain substances depending on the environmental situation: e.g.—a petrochemical plant that uses or synthesizes a limited number of chemical products, where rapid, low-level detection and identification of leaks or spills is a major safety, economic, or liability consideration. Therefore, even without the selectivity associated with chromatographic separation or antibody based cleanup, it is possible to get a rapid MS-based assessment of the environment.

Pattern recognition based on pyrolysis mass spectra of a large variety of chemical, biological, and mixed materials can be used for rapid, generic detection and classification. In one example, a bio-insecticide sample containing 90% of a pure chemical filler and 10% of the bio-insecticide plotted into the space between examples of pure bacteria and spectra for the filler. In this case, the pattern recognition approach was multilinear discriminant analysis rather than ANNs. Multilinear methods that can produce a score plot for visualization of sample similarities and differences provide a preferred basis for pattern recognition for this kind of problem. The ANNs, being so powerful, would generate a long list of “none-of-the-above” identifications (not very informative) when the samples were previously unseen mixtures of chemicals or bacteria whose pure spectra were in the database.

For situations in which a larger than usual amount of unrecognizable dust or chemical vapors enters the MS (directly, or through liquid concentration, thermal desorption, or impaction sampling) a total intensity threshold is set in the mass spectrometer to report an anomaly and generate a safety alarm. The same kind of threshold is also set for particular ions associated with anticipated hazardous chemicals or other likely contaminants. In this way the system can monitor the environment and yield rapid, useful warning even when the chemicals are not yet concentrated or separated for unequivocal identification and even when there is no basis for anticipating a particular problem.

### Other Embodiments

By way of example, but not by way of limitation, examples of further embodiments of the inventive method include:
clinical applications for general typing similar to Example 1, in which the advantages are chiefly speed and cost per analysis for characterizing a mixture of similar strains
clinical applications for rapid, precise typing of individual strains using the cell sorting option of Example 2. With the MS and pattern recognition, this can provide . . .
   almost exact matches with highly similar library strains,
   enough specificity to identify sources of noscomial infection,
   enough specificity to classify bacteria for antibiotic sensitivity and so suggest appropriate antibiotic treatment regimens, reducing shotgun or overkill prescription that leads to increased antibiotic resistance, a major health hazard.
law enforcement applications
   with enough specificity for general forensics, or
   toxicant determination.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

What is claimed is:

1. A method of testing for microorganisms in a sample taken from a non-laboratory source or environment, said method comprising the steps of:
(a) removing particulates from said sample;
(b) determining whether at least a threshold level of viable cells, non-viable cells, or a combination thereof is present in said sample; and
(c) determining, when at least said threshold level of viable cells, nonviable cells, or a combination thereof is determined to be present in said sample in step (b), whether at least one targeted microorganism is present in said sample.

2. The method of claim 1 wherein step (b) comprises:
adding to at least a portion of said sample a DNA-attaching dye effective for attaching to DNA in both said viable cells and said nonviable cells and
determining a level of said viable cells and said nonviable cells in said sample using flow cytometry to detect a signal emission of said DNA-attaching dye.

3. The method of claim 1 wherein step (b) comprises:
adding to at least a portion of said sample a DNA-attaching dye which is effective for attaching to DNA in said nonviable cells but will not substantially penetrate into said viable cells and
determining a level of said nonviable cells in said sample using flow cytometry to detect a signal emission of said DNA attaching dye.

4. The method of claim 1 wherein step (c) comprises:
adding to at least a portion of said sample a tag material effective for antibody-selective attachment to said targeted microorganism and
determining, at least preliminarily, whether at least a threshold level of said targeted microorganism is present in said sample using flow cytometry to detect said tag material.

5. The method of claim 4 wherein, when said targeted microorganism is determined, at least preliminarily, in step (c) to be present in said sample in at least said threshold level of said targeted microorganism, said method further comprises the step of (d) confirming whether said targeted microorganism is present in said sample by:
(i) recovering one or more cells from at least a portion of said sample;
(ii) culturing said one or more cells recovered in step (i) to produce cultured cells;
(iii) analyzing said cultured cells by mass spectrometry to obtain a spectral fingerprint for said cultured cells; and
(iv) determining whether said spectral fingerprint corresponds to said targeted microorganism.

6. The method of claim 5 wherein, in step (iv), artificial neural network, multi-linear statistical, expert system, correlation analysis or other pattern recognition is used to determine whether said spectral fingerprint corresponds to said targeted microorganism.

7. The method of claim 5 wherein said spectral fingerprint is drift compensated prior to determining whether said spectral fingerprint corresponds to said targeted microorganism.

8. The method of claim 5 wherein step (i) comprises recovering said one or more cells from said portion of said sample by ImmunoMagnetic Separation using an anchored antibody material selective for said targeted microorganism or for a genus, species, subspecies, serotype, or strain including said targeted microorganism.

9. The method of claim 1 further comprising the steps, prior to step (a), of:
dividing said sample into a plurality of portions and labeling each of said portions with a bar code including a sample identification code and a task code.

10. The method of claim 1 wherein said sample is taken from a food product.

11. The method of claim 1 wherein said sample is taken from a food processing facility.

12. The method of claim 1 wherein said sample is taken from a medical patient.

13. The method of claim 1 wherein said sample is taken from a medical treatment facility.

14. A method of testing for microorganisms in a sample taken from a non-laboratory source or environment, said method comprising the steps of:
(a) removing particulates from said sample;
(b) adding to at least a portion of said sample a first DNA-attaching dye of a type effective for attaching to DNA in viable cells and nonviable cells;
(c) adding to at least a portion of said sample a second DNA-attaching dye of a type effective for attaching to DNA in said nonviable cells but which will not substantially penetrate into said viable cells;
(d) determining a level of said viable cells and a level of said nonviable cells in said sample by flow cytometry based upon signal emissions of said first and said second DNA-attaching dyes;
(e) adding to at least a portion of said sample a tag material effective for antibody selective attachment to a targeted microorganism; and
(f) determining, at least preliminarily, whether at least a threshold level of said targeted microorganism is present in said sample by flow cytometry based upon a signal emission of said tag material.

15. The method of claim 14 wherein steps (d) and (f) are conducted simultaneously.

16. The method of claim 14 wherein, when said targeted microorganism is determined, at least preliminarily, to be present in said sample at least said threshold level and in the event that at least a threshold level of said viable cells is determined to be present in said sample, said method further comprises the step of (g) confirming whether said targeted microorganism is present in said sample by mass spectrometry.

17. The method of claim 16 wherein step (g) comprises:
(i) recovering one or more cells from at least a portion of said sample;
(ii) culturing said one or more cells recovered in step (i) to produce cultured cells;
(iii) analyzing said cultured cells by mass spectrometry to obtain a spectral fingerprint for said cultured cells; and
(iv) determining whether said spectral fingerprint corresponds to said targeted microorganism.

18. The method of claim 17 wherein step (i) comprises recovering one or more cells by ImmunoMagnetic Separation using an anchored antibody material selective for said targeted microorganism or for a genus, species, subspecies, serotype, or strain including said targeted microorganism.

19. The method of claim 17 wherein in step (iv), artificial neural network, multi-linear statistical, expert system, correlation analysis or other pattern recognition is used to determine whether said spectral fingerprint corresponds to said targeted microorganism.

20. The method of claim 19 wherein said spectral fingerprint is drift compensated prior to determining whether said spectral fingerprint corresponds to said targeted microorganism.

21. The method of claim 14 further comprising the steps, prior to steps (a)-(f), of:
dividing said sample into a plurality of portions and
labeling each of said portions with bar code including an identification code and a task code.

22. The method of claim 14 wherein said sample is taken from a food product.

23. The method of claim 14 wherein said sample is taken from a food processing facility.

24. The method of claim 14 wherein said sample is taken from a medical patent.

25. The method of claim 14 wherein said sample is taken from a medical treatment facility.

26. A method of testing for microorganisms in a sample taken from a non-laboratory source or environment, said method comprising the steps of:
(a) removing particulates from said sample;
(b) adding to at least a portion of said sample a DNA-attaching dye of a type effective for attaching to DNA in nonviable cells but which will not substantially penetrate into viable cells;
(c) adding to said portion of said sample a tag material effective for antibody selective attachment to a targeted microorganism; and
(d) determining, at least preliminarily, whether at least a threshold level of viable cells of said targeted microorganism is present in said sample by flow cytometry based upon signal emissions of said DNA-attaching dye and said tag material.

27. The method of claim 26 wherein, when said threshold level of viable cells of said targeted microorganism is determined to be present in said sample, said method further comprises the step of (e) confirming whether said targeted microorganism is present in said sample by mass spectrometry.

28. The method of claim 27 wherein step (e) comprises:
(i) recovering one or more cells from at least a portion of said sample;
(ii) culturing said one or more cells recovered in step (i) to produce cultured cells;
(iii) analyzing said cultured cells by mass spectrometry to obtain a spectral fingerprint for said cultured cells; and
(iv) determining whether said spectral fingerprint corresponds to said targeted microorganism.

29. The method of claim 28 wherein step (i) comprises recovering one or more cells by ImmunoMagnetic Separation using an anchored antibody material selective for said targeted microorganism or for a genus, species, subspecies, serotype, or strain including said targeted microorganism.

30. The method of claim 28 wherein in step (iv), artificial neural network, multi-linear statistical, expert system, correlation analysis, or other pattern recognition is used to determine whether said spectral fingerprint corresponds to said targeted microorganism.

31. The method of claim 30 wherein said spectral fingerprint is drift compensated prior to determining whether said spectral fingerprint corresponds to said targeted microorganism.

32. The method of claim 26 further comprising the steps, prior to steps (a)-(d), of:
dividing said sample into a plurality of portions and
labeling each of said portions with a bar code including an identification code and a task code.

33. The method of claim 26 wherein said sample is taken from a food product.

34. The method of claim 26 wherein said sample is taken from a food processing facility.

35. The method of claim 26 wherein said sample is taken from a medical patient.

36. The method of claim 26 wherein said sample is taken from a medical treatment facility.

37. A method of testing for microorganisms in a sample taken from a non-laboratory source or environment, said method comprising the steps of:
(a) removing particulates from said sample;
(b) recovering one or more cells from at least a portion of said sample by flow cytometry sorting and
(c) determining whether said one or more cells recovered in step (b) is/are a targeted microorganism.

38. The method of claim 37 wherein, prior to step (b), said one or more cells is/are tagged with an antibody material selective for attachment to said targeted microorganism.

39. The method of claim 37 wherein said one or more cells is/are recovered in step (b) by said flow cytometry sorting based upon a selected cell morphology.

40. The method of claim 39 wherein said one or more cells is/are sorted by said flow cytometry sorting based upon forward and side light scattering characteristics.

41. The method of claim 37 wherein a mass spectrometry analysis is used in step (c) to determine whether said one or more cells recovered in step (b) is/are said targeted microorganism.

42. The method of claim 41 further comprising the step, prior to step (c), of culturing said one or more cells recovered in step (b).

43. The method of claim 37 wherein said sample is taken from a food product.

44. The method of claim 37 wherein said sample is taken from a food processing facility.

45. The method of claim 37 wherein said sample is taken from a medical patient.

46. The method of claim 37 wherein said sample is taken from a medical treatment facility.

47. A method of monitoring air comprising the steps of:
(a) concentrating particles of selected dimensions from said air;
(b) placing at least a portion of said particles concentrated in step (a) into a liquid suspension;
(c) analyzing said liquid suspension by mass spectrometry to obtain a spectral fingerprint of said particles; and
(d) identifying said particles based upon said spectral fingerprint.

48. The method of claim 47 wherein said particles are identified in step (d) by multilinear discriminant analysis.

49. The method of claim 47 wherein said particles are identified in step (d) by artificial neural network pattern recognition.

50. The method of claim 47 further comprising the steps of:
(e) capturing a chemical vapor in said air by filtration;
(f) desorbing said chemical vapor captured in step (e) to produce a solution, a vapor, or a pyrolysate for analysis;
(g) analyzing said solution, said vapor, or said pyrolysate by mass spectrometry to obtain a spectral fingerprint of said chemical vapor; and
(h) identifying said chemical vapor based upon said spectral fingerprint of said chemical vapor.

51. The method of claim 50 wherein said chemical vapor is identified in step (h) by multilinear discriminant analysis.

52. The method of claim 50 wherein said chemical vapor is identified in step (h) by artificial neural network pattern recognition.

53. A method of monitoring air comprising the steps of:
(a) capturing a chemical vapor in said air by filtration;
(b) desorbing said chemical vapor captured in step (a) to produce a solution, a vapor, or a pyrolysate for analysis;
(c) analyzing said solution, said vapor, or said pyrolysate by mass spectrometry to obtain a spectral fingerprint of said chemical vapor; and
(d) identifying said chemical vapor based upon said spectral fingerprint.

54. The method of claim 53 wherein said chemical vapor is identified in step (d) by multilinear discriminant analysis.

55. The method of claim 53 wherein said chemical vapor is identified in step (d) by artificial neural network pattern recognition.
